SEQUENCE LISTING (3 sequences)

SEQ ID NO: 1 - DNA, 52 bp, artificial sequence
  Description of Artificial Sequence Synthetic oligonucleotide
gtttgctgacactgatcgcactctgatatgtggagggagggctgagggtttg

SEQ ID NO: 2 - DNA, 52 bp, artificial sequence
  Description of Artificial Sequence Synthetic oligonucleotide
gtttgctgacactgatcgcactctgatatgtggggtgggcctatgacagggt

SEQ ID NO: 3 - DNA, 13 bp, artificial sequence
  Description of Artificial Sequence Synthetic oligonucleotide
cagcaaactgttt

(19) United States
(12) Patent Application Publication
(10) Pub. No.: US 2022/0298505 A1
(43) Pub. Date: Sep. 22, 2022

(54) METHOD FOR CONSTRUCTING PACBIO SEQUENCING LIBRARY

(71) Applicant: BERRY GENOMICS CO., LTD, Beijing (CN)

(72) Inventors: Jianguang Zhang, Beijing (CN); Haiman Zhang, Beijing (CN); Xiaojie Zhang, Beijing (CN); Bo Shi, Beijing (CN); Aiping Mao, Beijing (CN)

(21) Appl. No.: 17/636,762

(22) PCT Filed: Jun. 22, 2020
(86) PCT No.: PCT/CN2020/097545

§ 371 Date: Feb. 18, 2022

(30) Foreign Application Priority Data
Jun. 26, 2019 (CN) 201910560767.2

(51) Int. Cl. C12N 15/10

(52) U.S. Cl. CPC C12N 15/1068

(57) ABSTRACT

Provided in the present invention is a method for constructing a PacBio sequencing library, comprising the following steps: (1) obtaining a target double-stranded DNA; (2) adding a thermostable RNA ligase to respectively connect two ends of the double-stranded DNA to form a closed loop to obtain a dumbbell-shaped DNA library; (3) purifying the dumbbell-shaped DNA library; and (4) binding with sequencing primers and adding a DNA polymerase to obtain a PacBio sequencing library.

### TECHNICAL FIELD

The disclosure relates to a method for constructing a PacBio sequencing library. In particular, The disclosure relates to a method for rapidly constructing a PacBio sequencing library by utilizing the properties of a double-stranded DNA and a thermostable RNA ligase under a high temperature.

### BACKGROUND

Next-generation sequencing technologies, which have become increasingly mature in recent years, are widely used in clinical research due to their outstanding advantages such as high throughput, high accuracy, high sensitivity, high automation and low operating costs. With the rapid development of sequencing technologies, third-generation sequencing technologies have also emerged, including SMRT technology from Pacific Biosciences (hereafter referred to as PacBio) ¹, nanopore single molecule technology from company Oxford Nanopore Technologies ² and Heliscope technology from company Helicos ³. Compared to the previous two generations of sequencing technologies, their most important feature is single-molecule long-fragment sequencing, wherein SMRT technology and Heliscope technology use fluorescent signals for sequencing, while nanopore single-molecule sequencing technology uses electrical signals generated by different bases for sequencing. Because the third-generation sequencing technology does not require a PCR amplification, the sequencing reaction speed is fast and the bias for GC bases is low. However, a single-base sequencing is less accurate. The sequencing libraries of PacBio have a dumbbell-shaped structure, sequencing DNA polymerase can amplify the target fragment of the library in multiple rounds, and the results of multiple rounds of sequencing can be mutually calibrated. Thus, the accuracy of PacBio sequencing after calibration is high, and the accuracy of 10 Kb target fragment can reach 99.99%.

The construction of a dumbbell-shaped PacBio sequencing library generally includes the following steps: (1) obtaining a target double-stranded DNA; (2) repairing and filling ends of the DNA; (3) ligating with PacBio linkers; (4) purifying the DNA; (5) repairing the DNA; (6) removing unligated linkers and the target DNA by exonuclease digestion; (7) removing linkers by two-step purification; (8) adding sequencing primers to anneal and DNA polymerase to form PacBio sequencing libraries. Depending on the characteristics of the target double-stranded DNA, the step (5) may be omitted. Traditional PacBio sequencing library construction is tedious, time-consuming and inefficient.

### SUMMARY

In view of above, the disclosure provides a method for constructing a PacBio sequencing library. Specifically, the method of the disclosure for constructing a PacBio sequencing library comprises four steps of obtaining a target double-stranded DNA, respectively connecting two ends of the double-stranded DNA to form a closed loop, purifying the DNA, combining the sequencing primers and adding a DNA polymerase, preferably consisting of the above four steps. Thermostable RNA ligases, with the property of ligating single-stranded ssDNA, include Thermus bacteriophage RNA ligases ^{4, 5}, archaebacterium RNA ligases such as Methanobacterium thermoautorophicum RNA ligase 1 ⁶ and the like. Under a high temperature, the ends of double-stranded DNA are unlocked by respiration to form single strands ⁷, and thermostable RNA ligases can respectively connect the 5′ phosphate and 3′ hydroxyl linkages at the ends of two single-stranded DNA into a loop, to form a dumbbell-shaped DNA library structure. In combination with specific sequencing primers and sequencing DNA polymerases, this library can be applied to PacBio sequencing platform for sequencing (FIG. 1). The method involved in this application is simple and efficient, and the quality of PacBio libraries is reliable and reproducible, which facilitates the application of PacBio sequencing technology for clinical testing. With specific PCR amplification products, the disclosure can be applied to mutation detection of target DNA sequences. For sequences that are difficult to amplify by PCR, the target DNA sequences can be obtained by CRISPR/Cas9 cleavage of double-stranded DNA and other techniques, and the target DNA can be sequenced with specific sequencing primers using the technology of the disclosure.

Thus, the purpose of the disclosure is to solve the problem of complicated and inefficient construction of PacBio sequencing library at the current stage. After obtaining the target double-stranded DNA, the two ends of the DNA are respectively connected into a loop by a thermostable RNA ligase, and the dumbbell-shaped DNA library can be quickly obtained after purification. PacBio sequencing libraries are formed by binding sequencing primers complementary to terminal circular DNA, and binding with sequencing DNA polymerase.

Thus, in a first aspect, the present application provides a method of constructing a PacBio sequencing library, comprising the following steps: (1) obtaining a target double-stranded DNA, and optionally further purifying said target double-stranded DNA; (2) adding a thermostable RNA ligase to respectively connect two ends of said double-stranded DNA to form a closed loop to obtain a dumbbell-shaped DNA library; (3) purifying said dumbbell-shaped DNA library; and (4) binding with a sequencing primer and adding a DNA polymerase to obtain a PacBio sequencing library.

In one embodiment, the steps and reaction conditions for the specific construction of a PacBio sequencing library may vary and can be adjusted by those skilled in the art as needed. If the reaction system for obtaining the target double-stranded DNA in step (1) affects the reaction efficiency of the thermostable RNA ligase, it is necessary to add a step of purifying said double-stranded DNA after step (1). The purification method can be a magnetic bead-based or a silica membrane column-based method, and the like.

Under a high temperature, the thermostable RNA ligase has a high efficiency for respectively connecting the two ends of the DNA into a loop, and a dumbbell-shaped DNA with a high-purity can be directly obtained after purification. In one embodiment, if the sequence of the target double-stranded DNA in step (1) causes the thermostable RNA ligase to be inefficient in respectively connecting the two ends of the double-stranded DNA to form a closed loop, affecting the subsequent sequencing steps, then it is necessary to additionally treat with an exonuclease after step (2) so as to remove the non-dumbbell DNA.

According to an embodiment, said target double-stranded DNA is obtained by a PCR amplification, a multiplex PCR amplification, or a CRISPR/Cas9 cleavage.

In one embodiment, the double-stranded DNA is an HBB gene. In this embodiment, the primer sequences for PCR amplification are shown in SEQ ID NO: 1 and 2.

According to an embodiment, the sequences at both ends of said target double-stranded DNA are the same or different.

According to a preferred embodiment, the ends of said target double-stranded DNA are blund ends and/or sticky ends.

According to a preferred embodiment, the 5′ base at the end of the target double-stranded DNA has a phosphate group, and the 3′ base at the end of the target double-stranded DNA has a hydroxyl group. If the 5′ base at the end of said target double-stranded DNA does not have a phosphate group, the 5′ at the end of the target double-stranded DNA can be phosphorylation modified by a kinase such as T4 polynucleotide kinase.

According to the present application, the two ends of the target double-stranded DNA are respectively connected to form a closed loop with the thermostable RNA ligase, thereby forming a dumbbell-shaped DNA library. Specifically, the thermostable RNA ligase can be derived from commercial products (e.g., Lucigen's CircLigase II ssDNA Ligase, Cat # CL9021K) or a purified protein, i.e., selected from Thermus bacteriophage RNA ligase, an archaebacterium RNA ligase such as Methanobacterium thermoautorophicum RNA ligase 1 and the like. The conditions and methods for respectively connecting two ends of the target double-stranded DNA to form a closed loop can be adjusted by those skilled in the art according to actual needs. Said thermostable RNA ligase is incubated at a temperature suitable for said thermostable RNA to remain active, for a sufficient time to respectively connect the two ends of said double-stranded DNA to form a closed loop. For example, the target double-stranded DNA may be incubated at 40-70° C. suitable for thermostable RNA ligase activity for 30 minutes to 16 hours, so that the reaction of connecting the two ends to form a closed loop is fully carried out.

According to a preferred embodiment, said thermostable RNA ligase is a pre-adenylated thermostable RNA ligase.

The purpose of the purification in step (3) is primarily to remove the enzyme required for the reactions in steps (1) and (2) and the components of buffer solution. In one embodiment, the purification can be performed by a magnetic bead-based or a silica membrane column-based method, and the like.

According to a preferred embodiment, said circular DNA sequences at both ends of said dumbbell-shaped DNA library are the same or different. If the circular DNA sequences at the two ends are different, the corresponding sequencing primers can be designed according to the DNA sequence of one end of the two ends.

According to a preferred embodiment, said target double-stranded DNA has or does not have a Barcode, which can be decided by a person skilled in the art as necessary.

According to a preferred embodiment, the length of said sequencing primer which is inversely complementary to the 4 sequence at one end of said dumbbell-shaped DNA library is 6-40 nt. Preferably, the sequence of said sequencing primer is shown in SEQ ID NO: 3.

The method described in the present application is characterized in that the thermostable RNA ligase respectively connects two ends of the double-stranded DNA to form a closed loop in the range of 40-70° C., and which facilitates the rapid construction of a PacBio sequencing library.

A second aspect of the present application also provides a kit, said kit is used for constructing a PacBio sequencing library by the method according to the first aspect of the present application.

According to a preferred embodiment, said kit comprises (a) one or more reagents selected from the group consisting of an amplification primer for the target double-stranded DNA or CRISPR/Cas9 reagent, a thermostable RNA ligase, a sequencing primer, and a DNA polymerase; and (b) an instruction.

The superior technical effect of the method described in the present application lies mainly in the following aspects:

(1) Simple and rapid experimental procedure. After obtaining the target double-stranded DNA, it is only necessary to use the thermostable RNA ligase to respectively connect the two ends of the double-stranded DNA to form a closed loop and then the dumbbell-shaped DNA library structure can be obtained.

(2) High reaction efficiency. Under the high temperature condition, the thermostable RNA ligase has a high efficiency for connecting the two ends of the DNA to form a closed loop, so the step of exonuclease digestion to remove the un-looped DNA can be omitted and the high-purity dumbbell-shaped DNA can be directly obtained after purification.

(3) High flexibility of target double-stranded DNA ends and the sequencing primer. Under a high temperature, the target double-stranded DNA ends are partially melted due to respiration. The 5′ phosphate group and 3′ hydroxyl group of the two ends of double-stranded DNA are respectively connected by the action of thermostable RNA ligase to respectively form a closed loop structure, and the reverse complementary sequencing primer can be designed. Taking PCR as an example, if only a single target region is detected, a sequencing primer that is reverse complementary to the end of the target region can be designed; if multiple target regions are detected simultaneously using multiplex PCR, the same sequence can be added to the end of the PCR primer to facilitate the design of reverse complementary sequencing primers.

### DETAILED DESCRIPTION

The disclosure will be described in detail below with reference to examples. It should be noted that those skilled in the art should understand that the examples of The disclosure are only for the purpose of illustration, and do not constitute any limitation to The disclosure.

### BRIEF DESCRIPTION OF THE FIGURES

FIG. 1 is a schematic diagram of the principle of rapidly constructing a PacBio sequencing library, which illustrates the process of rapidly constructing a PacBio sequencing library.

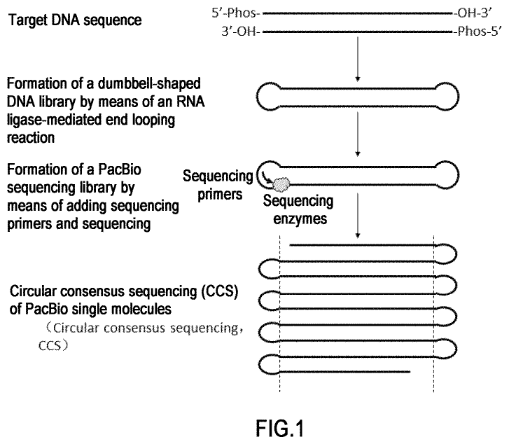

FIG. 2 is a DNA gel diagram of the HBB gene mutation sample amplified according to the PCR method in Example 1, which shows the PCR amplification product of the HBB gene.

FIG. 3 is the PacBio sequencing result of the HBB gene heterozygous mutation IVS-II-654 (C-T) sample (the antisense strand of the HBB gene is shown in the figure). Sequencing of this sample yielded 896 sequenced molecules covering the HBB gene region, of which 399 sequenced molecules were detected at the arrow position with a G signal and no IVS-II-654 (C-T) type mutation, and the other 497 sequenced molecules were detected at the arrow position with an A signal and an IVS-II-654 (C-T) type mutation, indicating that this sample is IVS-II-654 (C-T) heterozygous mutation sample.

### EXAMPLES

### Example 1. Construction of a PacBio Sequencing Library for Detection of HBB Gene Mutations According to the Method of the Disclosure

### Step 1: PCR Amplification of the HBB Gene.

200 μL of human peripheral blood was collected with an EDTA anticoagulant tube. The reaction system was prepared according to the following table (wherein the 16 bases marked with an underline are the Barcode sequence bcl001 provided by the PacBio company. If there are multiple samples, different Barcodes can be used for each sample).

| | |
|---|---|
| 2x MightyAmp Buffer Ver.2 | 25.0 μl |
| Primer HBB-F (10 uM) | 1.0 μl |
| 5′phos-GTTTGCTGACACTGATC | |
| GCACTCTGATATGTGGAGGGAGGGCTGAGG | |
| GTTTG-3' (SEQ ID NO: 1) | |
| Primer HBB-R (10uM) | 1.0 μl |
| 5'phos-GTTTGCTGACACTGATC | |
| GCACTCTGATATGTGGGGTGGGCCTATGACA | |
| GGGT-3' (SEQ ID NO: 2) | |
| ddH₂O | 21.0 μl |
| MightyAmp DNA polymerase (Takara, | 1.0 μl |
| Cat#R071Q) | |
| human peripheral blood | 1.0 μl |

On the PCR instrument, the amplification was performed under the following conditions:

| | | |
|---|---|---|
| Temperature | Time | Cycles |

| | | | |
|---|---|---|---|
| 98° C. | 60 | sec | 1 |
| 98° C. | 10 | sec | 28 |
| 63° C. | 15 | sec | |
| 68° C. | 3 | min | |
| 68° C. | 5 | min | 1 |

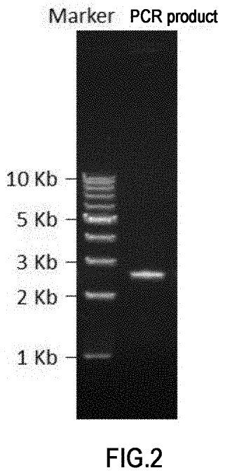

After amplification was completed, Qubit dsDNA BR reagent (ThermoFisher, Cat # Q32850) was used to determine DNA concentration on a Qubit 3 Fluoromter (ThermoFisher, Cat # Q33216), and ddH₂O was used to dilute the amplification product to 100 ng/μl. The PCR amplification product was verified with a DNA agarose gel (FIG. 2).

### Step 2: Construction of the Dumbbell-Shaped DNA Library Using a Thermostable RNA Ligase.

The reaction system was prepared as indicated in the following table.

| | | |
|---|---|---|
| PCR products (100 ng/ul) | 10.0 | μl |
| CircLigase II 10xReaction Buffer | 2.0 | μl |
| MnCl₂ (50 mM) | 1.0 | μl |
| Betaine (5M) | 4.0 | μl |
| ddH₂O | 2.0 | μl |
| CircLigase II ssDNA Ligase (100 U) (Lucigen, CL9021K) | 1.0 | μl |

On a PCR instrument, the reaction system was reacted at 60° C. for 1 hour.

### Step 3: Purification of the Dumbbell-Shaped DNA.

After step 2 was completed, 0.6× Ampure PB magnetic beads (Pacbio, Cat #100-265-900) were used to purify twice according to the manufacturer's instruction, and finally, 10 μl Elution Buffer was used for DNA elution. The obtained DNA Elution Solution is the target DNA dumbbell-shaped DNA library. The DNA concentration determined on a Qubit 3 Fluoromter (ThermoFisher, Cat # Q33216) using Qubit dsDNA HS reagent (ThermoFisher, Cat # Q32851) was 43.4 ng/μl.

### Step 4: Preparation of a PacBio Sequencing Library.

### 1) Annealing a Sequencing Primer to the Dumbbell-Shaped DNA.

The reaction system was prepared as indicated in following table.

| | |
|---|---|
| Step 3 dumbbell-shaped DNA library | 6.0 μl |
| (83.4 ng/μl) | |
| Sequencing Primer (100 uM) | 1.0 μl |
| 5-CAGCAAACTGTTT-3 (SEQ ID NO: 3) | |
| (underlined and bolded bases were 2′ | |
| methoxy modified) | |
| TrisHCl (10 mM, pH8.0) | 3.0 μl |

On the PCR instrument, the amplification was performed under the following conditions:

| | | |
|---|---|---|
| Temperature | Time | Cycles |

| | | | | |
|---|---|---|---|---|
| 98° | C. | 60 | sec | 1 |
| 95° | C. | 3 | min | 1 |
| 70° | C. | 5 | min | 1 |
| 65° | C. | 5 | min | 1 |
| 60° | C. | 5 | min | 1 |
| 55° | C. | 5 | min | 1 |
| 50° | C. | 5 | min | 1 |
| 45° | C. | 5 | min | 1 |
| 40° | C. | 5 | min | 1 |
| 35° | C. | 5 | min | 1 |
| 30° | C. | 5 | min | 1 |
| 25° | C. | 5 | min | 1 |

| | | | |
|---|---|---|---|
| 4° | C. | Forever | 1 |

As the reaction was completed, 1.5× Ampure PB magnetic beads (PacBio, Cat #100-265-900) were used to purify twice according to the manufacturer's instruction and the DNA was finally eluted with 10 ul Elution Buffer.

### 2) Sequencing Polymerase Binding Reaction.

The reaction system was prepared according to the following table, in which the reagents were obtained from Sequel II Binding and Internal Control 1.0 Kit (PacBio, Cat #101-731-100):

| | | |
|---|---|---|
| Sequel Binding Buffer | 40 | μl |
| DTT | 20 | μl |
| Sequel dNTP | 20 | μl |
| Step 1) annealed product | 6 | μl |
| Sequel II Polymerase 1.0 | 6 | μl |
| Total reaction volume | 92 | μl |

The reaction system was reacted at 30° C. for 1 hour on the PCR instrument, and then placed at 4° C. to form a PacBio sequencing library.

### 3) Purifying the PacBio Sequencing Library.

92 μl Ampure PB magnetic beads (PacBio, Cat #100-265-900) were added to the product of 2). Then, the PacBio sequencing library was purified according to the instructions of the PacBio SMRT 8.0, and finally was eluted by 101.1 μl Complex Dilution Buffer.

### 4) Sequencing the PacBio Library.

98.5 μl of the purified library in step 3) was added to 3.8 μl of Diluted Internal Control from Sequel II Binding and Internal Control 1.0 Kit (PacBio, Cat #101-731-100), 11.5 μl DTT and 1.2 μl Sequel Additive. After mixing evenly, the product was tested on Sequel II platform using SMRT Cell 8M sequencing chip (PacBio, Cat #101-389-001) and the sequencing reagent (PacBio, Cat #101-768-000), with CCS mode for 15 hours.

### Step 5: Analysis of Sequencing Results.

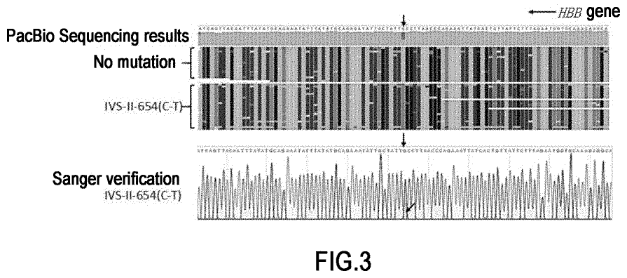

Representative sequencing results are presented in FIG. 3. The sample detected by The disclosure is a heterozygous mutation of HBB gene IVS-II-654 (C-T), which is consistent with the Sanger sequencing result.

It should be noted that although the above examples elucidate some features of The disclosure, they are not intend to limit the disclosure. Those skilled in the art know there can be various modifications and changes. The reaction reagents, reaction conditions and others involved in PacBio sequencing library construction can be adjusted and changed according to specific needs. Therefore, for those skilled in the art, without departing from the concept and principle of The disclosure, several simple substitutions can be made, and these should all be included within the protection scope of The disclosure.

### REFERENCES

[1]Roberts R J, et al. The advantages of SMRT sequencing. Genome Biol. 2013 Jul. 3; 14(7):405. Erratum in: Genome Biol. 2017 Aug. 16; 18(1):156. doi: 10.1186/gb-2013-14-6-405.
[2] Jain M, et al. Nanopore sequencing and assembly of a human genome with ultra-long reads. Nat Biotechnol. 2018 April; 36(4):338-345. doi: 10.1038/nbt.4060.
[3] Thompson J F, et al. Single molecule sequencing with a HeliScope genetic analysis system. Curr Protoc Mol Biol. 2010 October; Chapter 7:Unit7.10.
[4] Blondal T, et al. Isolation and characterization of a thermostable RNA ligase 1 from a Thermus scotoductus bacteriophage TS2126 with good single-stranded DNA ligation properties. Nucleic Acids Res. 2005 Jan. 7; 33(1):135-42. doi: 10.1093/nar/gki149.
[5] Blondal T, et al. Discovery and characterization of a thermostable bacteriophage RNA ligase homologous to T4 RNA ligase 1. Nucleic Acids Res. 2003 Dec. 15; 31(24):7247-54. doi:10.1093/nar/gkg914.
[6] Torchia C, et al. Archaeal RNA ligase is a homodimeric protein that catalyzes intramolecular ligation of single-stranded RNA and DNA. Nucleic Acids Res. 2008 November; 36(19): 6218-6227. doi:10.1093/nar/gkn602.

[7] Altan-Bonnet G, et al. Bubble Dynamics in Double-Stranded DNA. Phys Rev Lett. 2003 Apr. 4; 90(13): 138101. doi: 10.1103/PhysRevLett.90.138101.

We claim:

1. A method of constructing a PacBio sequencing library, comprising the following steps:
(1) obtaining a target double-stranded DNA, and optionally further purifying said target double-stranded DNA;
(2) adding a thermostable RNA ligase to respectively connect two ends of said double-stranded DNA to form a closed loop to obtain a dumbbell-shaped DNA library;
(3) purifying said dumbbell-shaped DNA library; and
(4) binding with a sequencing primer and adding a DNA polymerase to obtain a PacBio sequencing library.

2. The method according to claim 1, wherein said target double-stranded DNA is obtained by a PCR amplification, a multiplex PCR amplification, or a CRISPR/Cas9 cleavage.

3. The method according to claim 1, wherein the sequences at both ends of said target double-stranded DNA are the same or different.

4. The method according to claim 1, wherein the 5′ base at the end of the target double-stranded DNA has a phosphate group, and the 3′ base at the end of the target double-stranded DNA has a hydroxyl group.

5. The method according to claim 1, wherein said target double-stranded DNA has or does not have a Barcode.

6. The method according to claim 1, wherein in said step (2), said thermostable RNA ligase is incubated at a temperature suitable for said thermostable RNA to remain active, for a sufficient time to respectively connect the two ends of said double-stranded DNA to form a closed loop.

7. The method according to claim 1, wherein said thermostable RNA ligase is selected from a Thermus bacteriobacteriophage RNA ligase and/or an archaebacterium RNA ligase.

8. The method according to claim 1, wherein said thermostable RNA ligase is a Methanobacterium thermoautotrophicum RNA ligase 1.

9. The method according to claim 1, wherein said thermostable RNA ligase is a pre-adenylated thermostable RNA ligase.

10. The method according to claim 1, wherein said purification in step (1) or (3) is carried out by a magnetic bead or silica gel membrane column.

11. The method according to claim 1, wherein said circular DNA sequences at both ends of said dumbbell-shaped DNA library are the same or different.

12. The method according to claim 1, wherein said sequencing primer is inversely complementary to the circular DNA sequence at one end of said dumbbell-shaped DNA library.

13. The method according to claim 1, wherein the length of said sequencing primer to inversely complementary to the circular DNA sequence at one end of said dumbbell-shaped DNA library is 6-40 nt.

14. The method according to claim 1, wherein the ends of said target double-stranded DNA are blund ends and/or sticky ends.

15. A kit used for constructing a PacBio sequencing library by the method according to claim 1.

16. The kit according to claim 15 , comprising
(a) one or more reagents selected from the group consisting of an amplification primer for the target double-stranded DNA or CRISPR/Cas9 reagent, a thermostable RNA ligase, a sequencing primer, and a DNA polymerase; and
(b) an instruction.
